(19) United States
(12) Patent Application Publication
(10) Pub. No.: US 2003/0103946 A1
(43) Pub. Date: Jun. 5, 2003

(54) Immunotherapeutic methods and molecules

(75) Inventors: Hans Josef Stauss, London (GB); Persis Jal Amrolia, London (GB)

Correspondence Address: PATREA L. PABST, HOLLAND & KNIGHT LLP, SUITE 2000, ONE ATLANTIC CENTER, 1201 WEST PEACHTREE STREET, N.E., ATLANTA, GA, 30309-3400, US

(73) Assignee: Imperial College Innovations Limited

(21) Appl. No.: 10/003,983

(22) Filed: Oct. 31, 2001

(30) Foreign Application Priority Data
Nov. 30, 2000 (WO) PCT/GB00/04566

(51) Int. Cl. A61K 48/00; C12Q 1/68; C07H 21/04; C12P 21/02; C07K 14/74; C07K 7/06; C07K 7/08; C12N 5/08

(52) U.S. Cl. 424/93.21; 435/6; 435/69.1; 530/329; 530/350; 530/328; 435/320.1; 435/372; 536/23.5

(57) ABSTRACT

A peptide comprising an HLA-binding peptide of human CD45 polypeptide or a portion or variant of said peptide provided that the peptide is not the intact human CD45 polypeptide.

Preferably, the peptide comprises the amino acid sequence FLYDVIAST or ALIAFLAFL or KLFTAKLNV or MIWEQKATV or NLSELHPYL or VNLSELHPYL or LLAFGFAFL or YLYNKETKL or LILDVPPGV or TLILDVPPGV or ILYNNHKFT or ILPYDYNRV or YILIHQALV or FQLHDCTQV or KLLAFGFAFL or YQYQYTNWSV or a portion or variant of any of these. Methods of leukaemia immunotherapy using specific cytotoxic T lymphocytes are also disclosed.

### CROSS-REFERENCE TO RELATED APPLICATIONS

This application claims priority under 35 U.S.C. §119 to International Application No. PCT/GB00/04566 filed Nov. 30, 2000.

The present invention relates to immunotherapeutic methods, and molecules and cells for use in immunotherapeutic methods. In particular, the present invention relates to the immunotherapy of leukaemia.

There is evidence that anti-tumour cytotoxic T lymphocytes (CTL) play an important role in vivo. Tumour reactive CTL have been shown to mediate tumour regression in animal models (Kast et al (1989) Cell 59, 603-614) and in man (Kawakami et al (1994) Proc. Natl. Acad. Sci. USA 91, 6458-6462). As with all types of anti-tumour therapy, a problem that needs to be overcome is that the therapy must destroy or inactivate the target tumour cells to a useful extent but that the therapy must not destroy or inactivate non-tumour cells to a deleterious extent. In other words, it is desirable if the therapy is selective for tumour cells to a beneficial extent.

Much of the current work on immunotherapy of cancer makes use of the fact that certain tumours express polypeptides which are not expressed in the equivalent non-tumour tissue, or makes use of the fact that the tumour expresses a mutant form of a polypeptide which is not expressed in the non-tumour tissue. However, it is not always possible to identify polypeptides in a tumour which fall into this category, and so other target polypeptides which can form the basis of an immunotherapeutic approach have been identified.

Work in melanoma patients has shown that peptide-epitopes recognised by melanoma-reactive CTL are frequently derived from tissue-specific differentiation antigens. Recognition of differentiation antigens which are expressed in normal tissues seems to violate the rules of immunological tolerance; however, the CTL recognition of melanoma-associated differentiation antigens could be explained by the fact that they are normally only expressed in melanocytes which exist in relative small numbers at immunologically privileged sites, thus failing to establish tolerance. There is also evidence that prostate-specific differentiation antigens can serve as targets for CTL against tumours of the prostate.

WO 00/26249 shows that WT-1 and gata-1 can serve as targets for CTL against leukaemia.

There remains, however, the need to develop new targets, and new methods of, immunotherapy.

CD45 is a haematopoietic differentiation antigen that is expressed on the surface of most haematopoietic cells. CD45 is an abundant, highly glycosylated surface protein and different isoforms of the protein are generated by alternative splicing of the CD45 gene. All haematopoietic malignancies express CD45 at similar levels as normal cells. Hence, CD45 is an unsuitable target for conventional immunotherapy in the autologous setting. However, we now show that it is an attractive target for immunotherapy of haematological malignancies after stem cell transplantation from HLA-mismatched donors. According to the invention, allo-restricted CTL specific for CD45 epitopes presented by recipient HLA molecules will selectively eliminate recipient haematopoietic cells (malignant and normal cells) but not donor haematopoietic cells. Alternatively, but without being bound by any theory, it is conceivable that recipient malignant haematopoietic cells are more susceptible to CTL killing than normal haematopoietic cells. In this case, the CTL may preferentially kill malignant haematopoietic cells although they express similar CD45 levels as normal haematopoietic cells.

Using the allo-MHC-restricted CTL approach, we have identified peptide epitopes in CD45 which may be presented by HLA-A0201 class I molecules and displayed on the surface of leukaemia cells expressing these proteins endogenously. HLA-A0201 negative responder individuals were used as a source of CTL specific for peptides presented by HLA-A0201 class I to molecule, and this approach allows identification of HLA-A0201 presented peptides independent of possible tolerance of autologous CTL.

HLA-A0201 is the most common HLA-A haplotype.

For the avoidance of doubt, the terms HLA and MHC are used interchangeably in this specification.

To the inventor's knowledge, CD45 has not previously been considered for immunotherapy. In any event, the inventor has now shown that, surprisingly, the allorestricted approach is applicable though CD45 is expressed at similar levels in normal cells and cells with haematopoietic malignancies.

A first aspect of the invention provides a peptide comprising an HLA-binding peptide of human CD45 polypeptide or a portion or variant of said peptide provided that the peptide is not the intact human CD45 polypeptide.

Preferably, the portion or variant is one which binds an HLA molecule.

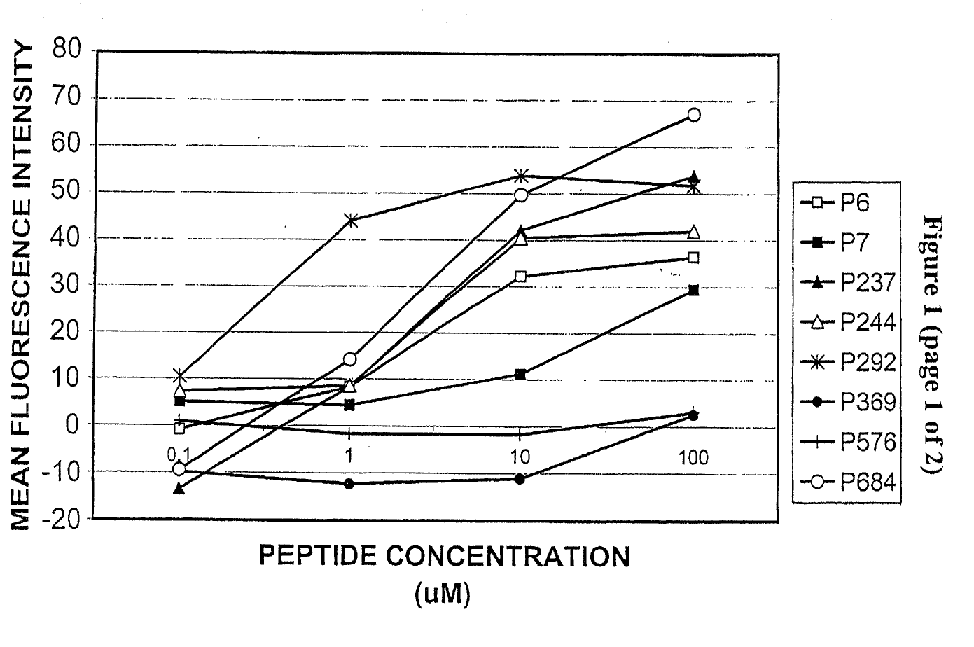

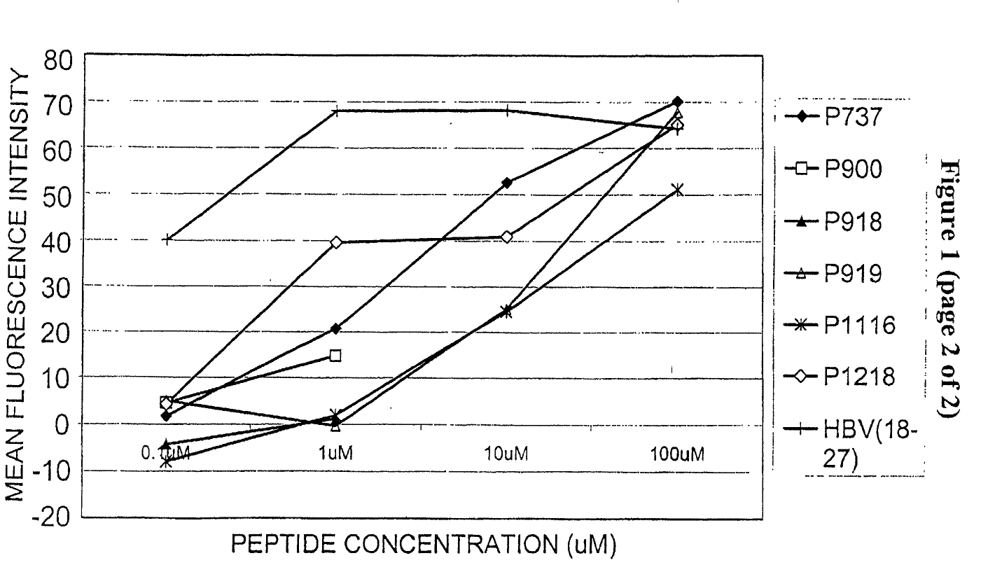

By “HLA-binding” peptide we mean a peptide which binds to one or more HLA molecules. Typically, a peptide will show HLA binding in a T2 binding assay when present in a concentration range of 10 μM to 1 nM. Whether or not a peptide of human CD45 is an HLA-binding peptide can be determined using methods known in the art. These methods include the T2 HLA stabilisation assay described in FIG. 1, and the method described by Elvin et al (1993) J. Immunol. Methods 158, 161-171.

The peptide may bind any one or more HLA molecule, but it is preferred if the peptide binds selectively to a particular HLA molecule. It is preferred that the peptide is one which binds HLA-A2 or HLA-A1 or HLA-A3 or HLA-B27. It is particularly preferred if the peptide binds to HLA-A0201.

By “peptide of human CD45 polypeptide” we mean a peptide which has contiguous amino acid sequence of the CD45 polypeptide. The peptide may be derived from the human CD45 polypeptide by chemical or enzymatic fragmentation, but it is preferred if the peptide is made by chemical synthesis or by expression using recombinant DNA technology as described below.

The sequence of CD45 is given in The Leukocyte Antigen Fact Book, 2^{nd} edition, page 244, Academic Press, Harcourt, Brace & Co (publishers), 1997.

In a preferred embodiment, the peptide is a peptide comprising the amino acid sequence FLYDVIAST or ALIAFLAFL or KLFTAKLNV or MIWEQKATV or NLSELHPYL or VNLSELHPYL or LLAFGFAFL or YLYNKETKL or LILDVPPGV or TLILDVPPGV or ILYNNHKFT or ILPYDYNRV or YILIHQALV or FQLHDCTQV or KLLAFGFAFL or YQYQYTNWSV or a portion or variant of any of these provided that the peptide is not the intact human CD45 polypeptide.

Preferably, the peptide of the invention comprises the amino acid sequence FLYDVIAST or a portion or variant thereof provided that the peptide is not intact human CD45 polypeptide.

In one embodiment of the invention, the peptide consists of a peptide having the amino acid sequence FLYDVIAST or ALIAFLAFL or KLFTAKLNV or MIWEQKATV or NLSELHPYL or VNLSELHPYL or LLAFGFAFL or YLYNKETKL or LILDVPPGV or TLILDVPPGV or ILYNNHKFT or ILPYDYNRV or YILIHQALV or FQLHDCTQV or KLLAFGFAFL or YQYQYTNWSV.

By “peptide” we include not only molecules in which amino acid residues are joined by peptide (—CO—NH—) linkages but also molecules in which the peptide bond is reversed. Such retro-inverso peptidomimetics may be made using methods known in the art, for example such as those described in Mézière et al (1997) J. Immunol. 159, 3230-3237, incorporated herein by reference. This approach involves making pseudopeptides containing changes involving the backbone, and not the orientation of side chains. Mézière et al (1997) show that, at least for MHC class II and T helper cell responses, these pseudopeptides are useful. Retro-inverse peptides, which contain NH—CO bonds instead of CO—NH peptide bonds, are much more resistant to proteolysis.

Similarly, the peptide bond may be dispensed with altogether provided that an appropriate linker moiety which retains the spacing between the Cα atoms of the amino acid residues is used; it is particularly preferred if the linker moiety has substantially the same charge distribution and substantially the same planarity of a peptide bond.

It will be appreciated that the peptide may conveniently be blocked at its N- or C-terminus so as to help reduce susceptibility to exoproteolytic digestion.

By a “portion” of the given amino acid sequence we mean at least six consecutive amino acids of the given sequence such that the portion is still able to bind to an HLA molecule in substantially the same way as a peptide consisting of the given amino acid sequence. Preferably, the peptide comprising the portion is able to bind to an HLA molecule in substantially the same way as a peptide consisting of the given amino acid sequence.

By a “variant” of the given amino acid sequence we mean that the side chains of, for example, one or two of the amino acid residues are altered (for example by replacing them with the side chain of another naturally occurring amino acid residue or some other side chain) such that the peptide is still able to bind to an HLA molecule in substantially the same way as a peptide consisting of the given amino acid sequence. For example, a peptide may be modified so that it at least maintains, if not improves, the ability to interact with and bind a suitable MHC molecule, such as HLA-A0201, and so that it at least maintains, if not improves, the ability to generate activated CTL which can recognise and kill cells which express a polypeptide which contains an amino acid sequence as defined in the first aspect of the invention (for example, CD45). Positions 2 and 9 of an HLA-A2-binding nonamer are typically anchor residues. Modifications of these and other residues involved in binding HLA-A2 may enhance binding without altering CTL recognition (for example, see Tourdot et al (1997) J. Immunol. 159, 2391-2398).

Those amino acid residues that are not essential to interact with the T cell receptor can be modified by replacement with another amino acid whose incorporation does not substantially affect T cell reactivity and does not eliminate binding to the relevant MHC.

Thus, the peptides of the invention are ones which, typically, selectively and reversibly bind one or more HLA molecules; most typically they are able to bind HLA-A0201.

The peptides of the invention may be of any size, but typically they may be less than 100 000 in molecular weight, preferably less than 80 000, more preferably less than 50 000 and typically about 40 000 or 30 000 or 20 000 or 10 000 or 5 000. In terms of the number of amino acid residues, the peptides of the invention may have fewer than 1000 residues, preferably fewer than 800 residues, more preferably fewer than 5000 residues or about 40 or 30 or 20 or 15 or 12 or 11 or 10 or 9 or 8 or 7 residues.

It will be appreciated from the following that in some applications the peptides of the invention may be used directly (ie they are not produced by expression of a polynucleotide in a patient's cell or in a cell given to a patient); in such applications it is preferred that the peptide has fewer than 100 residues, preferably fewer than 50 residues or about 40 or 30 or 20 or 15 or 12 or 11 or 10 or 9 or 8 or 7 residues.

It is preferred if the peptides of the invention are able to bind to HLA-A0201; however, the peptides may also bind to other HLA types as well as HLA-A0201. It is particularly preferred if the peptides bind selectively to HLA-A0201.

It is further preferred that the peptides of the invention are ones which can be used to generate peptide-specific CTL which show specific killing of T2 target cells when presenting the peptide (see Examples).

The peptides of the invention are particularly useful in immunotherapeutic methods to target and kill cells which express the CD45 polypeptide. The peptides may be derived from any portion of the CD45 protein, including portions that are unique to CD45 isoforms generated by alternative splicing.

Since these specific peptides consisting of the given amino acid sequences bind to HLA-A0201 it is preferred that the peptides of the invention are ones which bind HLA-A0201 and when so bound the HLA-A0201-peptide complex, when present on the surface of a suitable antigen-presenting cell, is capable of eliciting the production of a CTL which recognises a cell which expresses a polypeptide comprising the given amino acid sequence, such as the CD45 polypeptide.

It is well known that an optimum length for a peptide to bind to an HLA molecule is around 8 to 12 amino acid, preferably 9 amino acids.

A particular preferred peptide of the invention consists of the amino acid sequence FLYDVIAST.

If a peptide which is greater than around 12 amino acid residues is used directly to bind to a MHC molecule, it is preferred that the residues that flank the core HLA binding region are ones that do not substantially affect the ability of the peptide to bind to the MHC molecule or to present the peptide to the CTL. However, it will be appreciated that larger peptides may be used, especially when encoded by a polynucleotide, since these larger peptides may be fragmented by suitable antigen-presenting cells.

Peptides (at least those containing peptide linkages between amino acid residues) may be synthesised by the Fmoc-polyamide mode of solid-phase peptide synthesis as disclosed by Lu et al (1981) J. Org. Chem. 46, 3433 and references therein. Temporary N-amino group protection is afforded by the 9-fluorenylmethyloxycarbonyl (Fmoc) group. Repetitive cleavage of this highly base-labile protecting group is effected using 20% piperidine in N,N-dimethylformamide. Side-chain functionalities may be protected as their butyl ethers (in the case of serine threonine and tyrosine), butyl esters (in the case of glutamic acid and aspartic acid), butyloxycarbonyl derivative (in the case of lysine and histidine), trityl derivative (in the case of cysteine) and 4-methoxy-2,3,6-trimethylbenzenesulphonyl derivative (in the case of arginine). Where glutamine or asparagine are C-terminal residues, use is made of the 4,4′-dimethoxybenzhydryl group for protection of the side chain amido functionalities. The solid-phase support is based on a polydimethyl-acrylamide polymer constituted from the three monomers dimethylacrylamide (backbone-monomer), bisacryloylethylene diamine (cross linker) and acryloylsarcosine methyl ester (functionalising agent). The peptide-to-resin cleavable linked agent used is the acid-labile 4-hydroxymethyl-phenoxyacetic acid derivative. All amino acid derivatives are added as their preformed symmetrical anhydride derivatives with the exception of asparagine and glutamine, which are added using a reversed N,N-dicyclohexyl-carbodiimide/1-hydroxybenzotriazole mediated coupling procedure. All coupling and deprotection reactions are monitored using ninhydrin, trinitrobenzene sulphonic acid or isotin test procedures. Upon completion of synthesis, peptides are cleaved from the resin support with concomitant removal of side-chain protecting groups by treatment with 95% trifluoroacetic acid containing a 50% scavenger mix. Scavengers commonly used are ethanedithiol, phenol, anisole and water, the exact choice depending on the constituent amino acids of the peptide being synthesised. Trifluoroacetic acid is removed by evaporation in vacuo, with subsequent trituration with diethyl ether affording the crude peptide. Any scavengers present are removed by a simple extraction procedure which on lyophilisation of the aqueous phase affords the crude peptide free of scavengers. Reagents for peptide synthesis are generally available from Calbiochem-Novabiochem (UK) Ltd, Nottingham NG7 2QJ, UK. Purification may be effected by any one, or a combination of, techniques such as size exclusion chromatography, ion-exchange chromatography and (principally) reverse-phase high performance liquid chromatography. Analysis of peptides may be carried out using thin layer chromatography, reverse-phase high performance liquid chromatography, amino-acid analysis after acid hydrolysis and by fast atom bombardment (FAB) mass spectrometric analysis.

The peptide of the invention may be comprised within, or fused to, an HLA heavy chain molecule such that the peptide is joined to the HLA molecule by a suitable, flexible linker such that the peptide can occupy the peptide-binding groove of the HLA molecule. Fusion molecules of this type, although using a different peptide, have been synthesised by Mottez et al (1995) J. Exp. Med. 181, 493-502, incorporated herein by reference.

The invention also includes a library of HLA-binding peptides of human CD45 polypeptide wherein for each member of the library the type of HLA molecule it binds is recorded. The peptides may be stored in any suitable form in the library (eg frozen in solution or lyophilised) and the information concerning the type of HLA molecule bound by the peptide recorded in any suitable form, eg look-up table or computer record.

A second aspect of the invention provides a polynucleotide encoding a peptide as defined in the first aspect of the invention. The polynucleotide may be DNA or RNA and it may or may not contain introns so long as it codes for the peptide. Of course, it is only peptides which contain naturally occurring amino acid residues joined by naturally-occurring peptide bonds which are encodable by a polynucleotide.

A preferred polynucleotide of the invention is one which encodes an HLA heavy chain joined via a flexible linker to a peptide of the invention such that when the fusion molecule is expressed in a suitable cell the peptide binds the HLA peptide binding groove.

A third aspect of the invention provides an expression vector capable of expressing a polypeptide according to the first or aspect of the invention.

A fourth aspect of the invention provides a polypeptide fusion molecule which comprises an HLA heavy chain molecule joined via a flexible linker to an HLA binding peptide of CD45 such that the HLA binding peptide is able to occupy the peptide-binding groove of the HLA molecule. Preferably, the HLA binding peptide of CD45 is a peptide according to the first aspect of the invention. Fusion molecules of this type can be made by reference to Mottez et al (supra), incorporated herein by reference. The fusion molecule is useful when exposed on the surface of a cell since it mimics an HLA molecule loaded with said peptide.

A fifth aspect of the invention provides a polynucleotide encoding the polypeptide fusion molecule of the fourth aspect of the invention.

A sixth aspect of the invention provides an expression vector capable of expressing the fusion molecule.

A variety of methods have been developed to operably link polynucleotides, especially DNA, to vectors for example via complementary cohesive termini. For instance, complementary homopolymer tracts can be added to the DNA segment to be inserted to the vector DNA. The vector and DNA segment are then joined by hydrogen bonding between the complementary homopolymeric tails to form recombinant DNA molecules.

Synthetic linkers containing one or more restriction sites provide an alternative method of joining the DNA segment to vectors. The DNA segment, generated by endonuclease restriction digestion as described earlier, is treated with bacteriophage T4 DNA polymerase or E. coli DNA polymerase I, enzymes that remove protruding, 3′-single-stranded termini with their 3′-5′-exonucleolytic activities, and fill in recessed 3′-ends with their polymerizing activities.

The combination of these activities therefore generates blunt-ended DNA segments. The blunt-ended segments are then incubated with a large molar excess of linker molecules in the presence of an enzyme that is able to catalyze the ligation of blunt-ended DNA molecules, such as bacteriophage T4 DNA ligase. Thus, the products of the reaction are DNA segments carrying polymeric linker sequences at their ends. These DNA segments are then cleaved with the appropriate restriction enzyme and ligated to an expression vector that has been cleaved with an enzyme that produces termini compatible with those of the DNA segment.

Synthetic linkers containing a variety of restriction endonuclease sites are commercially available from a number of sources including International Biotechnologies Inc, New Haven, Conn., USA.

A desirable way to modify the DNA encoding the polypeptide of the invention is to use the polymerase chain reaction as disclosed by Saiki et al (1988) Science 239,487-491. This method may be used for introducing the DNA into a suitable vector, for example by engineering in suitable restriction sites, or it may be used to modify the DNA in other useful ways as is known in the art.

In this method the DNA to be enzymatically amplified is flanked by two specific primers which themselves become incorporated into the amplified DNA. The said specific primers may contain restriction endonuclease recognition sites which can be used for cloning into expression vectors using methods known in the art.

The DNA (or in the case of retroviral vectors, RNA) is then expressed in a suitable host to produce a polypeptide comprising the compound of the invention (eg peptide according to the first aspect or fusion molecule according to the fourth aspect). Thus, the DNA encoding the polypeptide constituting the compound of the invention may be used in accordance with known techniques, appropriately modified in view of the teachings contained herein, to construct an expression vector, which is then used to transform an appropriate host cell for the expression and production of the polypeptide of the invention.

The DNA (or in the case of retroviral vectors, RNA) encoding the polypeptide constituting the compound of the invention may be joined to a wide variety of other DNA sequences for introduction into an appropriate host. The companion DNA will depend upon the nature of the host, the manner of the introduction of the DNA into the host, and whether episomal maintenance or integration is desired.

Generally, the DNA is inserted into an expression vector, such as a plasmid, in proper orientation and correct reading frame for expression. If necessary, the DNA may be linked to the appropriate transcriptional and translational regulatory control nucleotide sequences recognised by the desired host, although such controls are generally available in the expression vector. The vector is then introduced into the host through standard techniques. Generally, not all of the hosts will be transformed by the vector. Therefore, it will be necessary to select for transformed host cells. One selection technique involves incorporating into the expression vector a DNA sequence, with any necessary control elements, that codes for a selectable trait in the transformed cell, such as antibiotic resistance. Alternatively, the gene for such selectable trait can be on another vector, which is used to co-transform the desired host cell.

Host cells that have been transformed by the recombinant DNA of the invention are then cultured for a sufficient time and under appropriate conditions known to those skilled in the art in view of the teachings disclosed herein to permit the expression of the polypeptide, which can then be recovered.

Many expression systems are known, including bacteria (for example E. coli and Bacillus subtilis), yeasts (for example Saccharomyces cerevisiae), filamentous fungi (for example Aspergillus), plant cells, animal cells and insect cells.

The vectors typically include a prokaryotic replicon, such as the ColE1 ori, for propagation in a prokaryote, even if the vector is to be used for expression in other, non-prokaryotic, cell types. The vectors can also include an appropriate promoter such as a prokaryotic promoter capable of directing the expression (transcription and translation) of the genes in a bacterial host cell, such as E. coli, transformed therewith. Suitable vectors are well known in the art.

The present invention also relates to a host cell transformed with a polynucleotide vector construct of the present invention. The host cell can be either prokaryotic or eukaryotic. Bacterial cells may be preferred prokaryotic host cells in some circumstances and typically are a strain of E. coli such as, for example, the E. coli strains DH5 available from Bethesda Research Laboratories Inc., Bethesda, Md., USA, and RR1 available from the American Type Culture Collection (ATCC) of Rockville, Md.), USA (No ATCC 31343). Preferred eukaryotic host cells include yeast, insect and mammalian cells, preferably vertebrate cells such as those from a mouse, rat, monkey or human fibroblastic and kidney cell lines. Yeast host cells include YPH499, YPH500 and YPH501 which are generally available from Stratagene Cloning Systems, La Jolla, Calif. 92037, USA. Preferred mammalian host cells include Chinese hamster ovary (CHO) cells available from the ATCC as CCL61, NIH Swiss mouse embryo cells NIH/3T3 available from the ATCC as CRL 1658, monkey kidney-derived COS-1 cells available from the ATCC as CRL 1650 and 293 cells which are human embryonic kidney cells. Preferred insect cells are Sf9 cells which can be transfected with baculovirus expression vectors. Transformation of host cells with DNA and vectors of the invention can be accomplished using methods known in the art.

It will be appreciated that certain host cells of the invention are useful in the preparation of the peptides or fusion molecules of the invention, for example bacterial, yeast and insect cells.

A seventh aspect of the invention provides a method of producing a peptide of the first aspect of the invention, the method comprising culturing host cells which contain a polynucleotide or expression vector which encodes the peptide and obtaining the peptide from the host cell or culture medium.

The peptides of the invention are used in the production of CTL specific for CD45 peptides and which are presented by particular HLA molecules, such as HLA-A0201.

Thus, further aspects of the invention provide compositions which comprise a peptide according to a first aspect of the invention which compositions are suitable for raising CTL specific for the peptides when presented by a particular HLA molecule. Such compositions are typically sterile and pyrogen free. Typically, for the generation of CTL the peptides are used in the range 200 μM to 1 nM.

Typically, the composition is an aqueous composition. In some instances it may be desirable to include a peptide-solubilising agent such as DMSO in the composition.

A still further aspect of the invention include a kit of parts comprising a peptide according to the invention and an antigen presenting cell. Preferred antigen presenting cells are described below. The kit of parts is useful in the preparation of activated CTL as described below. A yet still further aspect of the invention includes an antigen-presenting cell wherein its MHC Class I molecules are loaded with a peptide of the invention. These cells are suitably comprised in a library of cells wherein each member cell of the library is loaded with a different peptide of the invention. As described in more detail below, it is convenient if for each peptide (and each cell within the library) an indication is given as to which type of Class I MHC molecule the peptide binds.

A further aspect of the invention provides a method for producing activated cytotoxic T lymphocytes (CTL) in vitro, the method comprising contacting in vitro CTL with antigen-loaded human class I MHC molecules expressed on the surface of a suitable antigen-presenting cell for a period of time sufficient to activate, in an antigen specific manner, said CTL wherein the antigen is a peptide according to the first aspect of the invention.

In one embodiment the antigen is linked to the MHC Class I molecule heavy chain expressed on the surface of the antigen presenting cell with a suitable flexible linker such that the peptide can occupy the MHC Class I binding groove. Thus, the antigen presenting cells may be cells which contain a polynucleotide (eg expression vector) encoding the fusion polypeptide of the fourth aspect of the invention.

Suitably, the CTL are CD8⁺ cells but they may be CD4⁺ cells. The MHC class I molecules may be expressed on the surface of any suitable cell and it is preferred if the cell is one which does not naturally express MHC class I molecules (in which case the cell is transfected to express such a molecule) or, if it does, it is defective in the antigen-processing or antigen-presenting pathways. In this way, it is possible for the cell expressing the MHC class I molecule to be loaded substantially completely with a chosen peptide antigen before activating the CTL.

The antigen-presenting cell (or stimulator cell) typically has an MHC class I molecule on its surface and preferably is substantially incapable of itself loading said MHC class I molecule with the selected antigen. As is described in more detail below, the MHC class I molecule may readily be loaded with the selected antigen in vitro.

Conveniently, said antigen-presenting cell is a mammalian cell defective in the expression of a peptide transporter such that, when at least part of said selected molecule is a peptide, it is not loaded into said MHC class I molecule.

Preferably the mammalian cell lacks or has a reduced level or has reduced function of the TAP peptide transporter. Suitable cells which lack the TAP peptide transporter include T2, RMA-S and Drosophila cells. TAP is the Transporter Associated with antigen Processing.

Thus, conveniently the cell is an insect cell such as a Drosophila cell

The human peptide loading deficient cell line T2 is available from the American Type Culture Collection, 12301 Parklawn Drive, Rockville, Md. 20852, USA under Catalogue No CRL 1992; the Drosophila cell line Schneider line 2 is available from the ATCC under Catalogue No CRL 19863; the mouse RMA-S cell line is described in Karre and Ljunggren (1985) J. Exp. Med. 162, 1745, incorporated herein by reference.

In a preferred embodiment the stimulator cell is a T2 cell or an RMA-S cell or a Drosophila cell transfected with a nucleic acid molecule capable of expressing said MHC class I molecule. Although T2 and RMA-S cells do express before transfection HLA class I molecules they are not loaded with a peptide.

When the antigen presenting cells are one which express the HLA-peptide fusion molecule of the fourth aspect of the invention, it is not necessary that they lack TAP peptide transporter.

Mammalian cells can be transfected by methods well known in the art. Drosophila cells can be transfected, as described in Jackson et al (1992) proc. Natl. Acad. Sci. USA 89, 12117, incorporated herein by reference.

Conveniently said host cell before transfection expresses substantially no MHC class I molecules.

It is also preferred if the stimulator cell expresses a molecule important for T cell costimulation such as any of B7.1, B7.2, ICAM-1 and LFA 3.

The nucleic acid sequences of numerous MHC class I molecules, and of the costimulator molecules, are publicly available from the GenBank and EMBL databases.

It is particularly preferred if substantially all said MHC class I molecules expressed in the surface of said stimulator cell are of the same type.

HLA class I in humans, and equivalent systems in other animals, are genetically very complex. For example, there are more than 110 alleles of the HLA-B locus and more than 90 alleles of the HLA-A locus. Although any HLA class I (or equivalent) molecule is useful in this aspect of the invention, it is preferred if the stimulator cell presents at least part of the selected molecule in an HLA class I molecule which occurs at a reasonably high frequency in the human population. It is well known that the frequency of HLA class I alleles varies between different ethnic groupings such as Caucasian, African, Chinese and so on. At least as far as the Caucasian population is concerned it is preferred that HLA class I molecule is encoded by an HLA-A2 allele, or an HLA-A1 allele or an HLA-A3 allele or an HLA-B27 allele. HLA-A0201 is particularly preferred.

In a further embodiment, combinations of HLA molecules may also be used. For example, a combination of HLA-A2 and HLA-A3 covers 74% of the Caucasian population.

For therapeutic use of the CTL in the methods of the invention, allogeneic cells are used in the preparation of CTL and this method is described in detail in WO 97/26328, incorporated herein by reference. For example, in addition to Drosophila cells and T2 cells, other cells may be used to present antigens such as CHO cells, baculovirus-infected insects cells, bacteria, yeast, vaccinia-infected target cells. In addition plant viruses may be used (see, for example, Porta et al (1994) Virology 202, 449-955 which describes the development of cowpea mosaic virus as a high-yielding system for the presentation of foreign peptides.

When allogeneic cells are used in the preparation of CTL, the CTL are allo-MHC-restricted with respect to the peptides of the invention. Thus, typically, the CTL are from an individual who is negative for a particular HLA and the peptide is presented by that particular HLA molecule by the antigen-presenting cell. It is particularly preferred that the CTL are from a HLA-A0201 negative responder individual and that the peptide is presented by a HLA-A0201 class I molecule by the antigen-presenting cell.

Exogenously applied peptides may be linked to a HIV tat peptide to direct them into the MHC Class I pathway for presentation by CTL (see, for example, Kim et al (1997) J. Immunol. 159, 1666-1668).

The activated CTL which are directed against the peptides of the invention are useful in therapy. Thus, a further aspect of the invention provides activated CTL obtainable by the foregoing methods of the invention.

A still further aspect of the invention provides activated CTL which selectively recognise a cell which expresses a polypeptide comprising an amino acid sequence given in the first aspect of the invention. Typically, the cell is a cell which expresses CD45. Preferably, the CTL recognises the said cell by binding to the peptide as defined in the first aspect of the invention. Thus, typically, the activated CTL recognises human Class I MHC molecules expressed on the surface of an antigen presenting cell and loaded with a peptide according to the first aspect of the invention.

The activated CTL of the invention may be packaged and presented for use in medicine. In particular, the HLA haplotype and the CD45 T cell epitope recognised by the CTL may be indicated. The invention also includes a pharmaceutical preparation comprising an activated CTL of the invention and a pharmaceutically acceptable carrier. Typically, the carrier is sterile and pyrogen free.

The CD45 polypeptide is expressed in leukaemias, such as lymphoid and myeloid leukaemias, and in other haematological malignancies. The CD45 molecule is also expressed in normal cells of the haematopoietic system. Thus, as discussed in more detail below, CD45 based immunotherapy will be combined with allogeneic stem cell transplantation. For example, the targeting of CD45 peptide epitopes presented by HLA-A0201 is provided for patients undergoing stem cell transplantation from HLA-A0201-negative donors. In this case, donor cells repopulate the patients haematopoietic system. These donor cells are resistant to CTL specific for HLA-A0201 presented peptide epitopes. Hence, such CTL selectively kill malignant and normal haematopoietic cells of the patient, but not normal haematopoietic cells of the stem cell donor. The allo-restricted approach is therefore used to generate CTL specific for CD45 peptides presented by HLA-A0201. As discussed in more detail below, the TCR of such allo-restricted CTL can be used for gene therapy of patients who might benefit from CD45-specific, HLA-restricted CTL.

Thus, the CTL are useful in a method of killing target cells in a patient which target cells express a polypeptide comprising an amino acid sequence given in the first aspect of the invention (eg CD45) wherein the patient is administered an effective number of the activated CTL. Typically, the patient has undergone stem cell transplantation from an HLA-mismatched donor and is administered an effective number of activated CTL derived from the same donor as the stem cell transplantation or from a further HLA-mismatched donor.

The target cells which are destroyed by tie activated CTL are malignant and haematopoietic cells which express a polypeptide comprising an amino acid sequence given in the first aspect of the invention. Typically, the malignant and normal cells to be killed in the patient are haematopoietic cells which express CD45.

Hence, the CTL are not from the patient but are from another individual. Of course, it is preferred if the individual is a healthy individual. By “healthy individual” we mean that the individual is generally in good health, preferably has a competent immune system and, more preferably, is not suffering from any disease which can be readily tested for, and detected. In this embodiment, the CTL are derived from an individual whose HLA class I molecules are mismatched with those of the patient. Thus, it is preferred if the CTL are allo-restricted. Treatment with allo-restricted CTL is described in WO 97/26328, incorporated herein by reference.

In order to readily select suitable CTL for treating a particular individual, the CTL may be held in a library. Thus, a further aspect of the invention provides a library of activated CTL wherein each member of the library (1) recognises a CD45 peptide when presented by a particular, recorded HLA and (2) has its HLA haplotype recorded.

The activated CTL may be stored in any suitable way, for example by cryopreservation in medium containing serum and DMSO as is well known in the art.

The information concerning the CD45 peptide recognised, and the HLA haplotype may be recorded in any suitable form eg in a look-up table or on computer. Preferably, each CTL is selective for a CD45 peptide.

Although not wishing to be bound by any theories, we believe that the use of allorestricted CTL directed against human CD45 relies on the principle that T-cell tolerance against epitopes from normally expressed proteins is self HLA-restricted. Thus, for example, an HLA-A2− negative donor may recognise epitopes presented by HLA-A2, to which an HLA-A2− positive host is tolerant. CD45 is abundantly expressed in both malignant and normal cells of the haematopoietic lineage. Since it is expressed in a haematopoietic-specific fashion, we believe that CTL recognising CD45 should not cause toxicity to other host tissues (graft-versus-host disease). We believe that, by virtue of their allorestriction, such CTL should not kill the donor haematopoietic cells, which are HLA-A2 negative.

It is particularly preferred if the allorestricted CTL directed against human CD45 are administered to a patient who has previously had a haplo-identical transplantation for leukaemia (see Ann. NY Acad. Sci., 1999, Apr. 30, Vol 872 pp 351-361). Recent advances have made haplo-identical transplantation for leukaemia feasible. The allorestricted CTL approach discussed above is aimed at improving the graft-versus-leukaemia effect.

The methods of the invention include methods of adoptive immunotherapy.

The activated CTL contain a T cell receptor (TCR) which is involved in recognising cells which express the polypeptide. It is useful if the cDNA encoding the TCR is cloned from the activated CTL and transferred into a further CTL for expression.

The TCRs of CTL clones of the invention specific for the peptides of the first aspect of the invention are cloned. The TCR usage in the CTL clones is determined using (i) TCR variable region-specific monoclonal antibodies and (ii) RT-PCR with primers specific for Vα and Vβ gene families. A cDNA library is prepared from poly-A mRNA extracted from the CTL clones. Primers specific for the C-terminal portion of the TCR α and β chains and for the N-terminal portion of the identified Vα and β segments are used. The complete cDNA for the TCR α and β chain is amplified with a high fidelity DNA polymerase and the amplified products cloned into a suitable cloning vector. The cloned α and β chain genes may be assembled into a single chain TCR by the method as described by Chung et at (1994) Proc. Natl. Acad. Sci. USA 91, 12654-12658. In this single chain construct the VαJ segment is followed by the VβDJ segment, followed by the Cβ segment followed by the transmembrane and cytoplasmic segment of the CD3 ζ chain. This single chain TCR is then inserted into a retroviral expression vector (a panel of vectors may be used based on their ability to infect mature human CD8⁺ T lymphocytes and to mediate gene expression: the retroviral vector system Kat is one preferred possibility (see Finer et al (1994) Blood 83, 43). High titre amphotrophic retrovirus are used to infect purified CD8⁺ T lymphocytes isolated from the peripheral blood of tumour patients following a protocol published by Roberts et al (1994) Blood 84, 2878-2889, incorporated herein by reference. Anti-CD3 antibodies are used to trigger proliferation of purified CD8⁺ T cells, which facilitates retroviral integration and stable expression of single chain TCRs. The efficiency of retroviral transduction is determined by staining of infected CD8⁺ T cells with antibodies specific for the single chain TCR. In vitro analysis of transduced CD8⁺ T cells establishes that they display the same tumour-specific killing as seen with the allo-restricted CTL clone from which the TCR chains were originally cloned. Populations of transduced CD8⁺ T cells with the expected specificity may be used for adoptive immunotherapy of the tumour patients. Patients may be treated with in between 10⁸ to 10¹¹ (most likely 10⁹-10¹⁰) autologous, transduced CTL.

Other suitable systems for introducing genes into CTL are described in Moritz et al (1994) Proc. Natl. Acad. Sci. USA 91, 4318-4322, incorporated herein by reference. Eshhar et al (1993) Proc. Natl. Acad. Sci. USA 90, 720-724 and Hwu et al (1993) J. Exp. Med. 178, 361-366 also describe the transfection of CTL.

Thus, a further aspect of the invention provides a TCR which recognises a cell which expresses a polypeptide comprising an amino acid sequence given in the first aspect of the invention, the TCR being obtainable from the activated CTL. Thus, typically, the TCR recognise a cell of the haematopoietic lineage which expresses CD45.

As well as the TCR, functionally equivalent molecules to the TCR are included in the invention. These include any molecule which is functionally equivalent to a TCR which can perform the same function as a TCR. In particular, such molecules include genetically engineered three-domain single-chain TCRs as made by the method described by Chung et al (1994) Proc. Natl. Acad. Sci. USA 91, 12654-12658, incorporated herein by reference, and referred to above.

Typically, the TCR or a functionally equivalent molecule to the TCR, recognised human Class I MHC molecular expressed on the surface of an antigen-presenting cell and loaded with a peptide according to the first aspect of the invention.

The invention also includes a polynucleotide encoding the TCR or functionally equivalent molecule, and an expression vector encoding the TCR or functionally equivalent molecule thereof. Expression vectors which are suitable for expressing the TCR of the invention include viral vectors such as retroviral vectors, lentiviral vectors, adenoviral vectors, vaccinia vectors (including the replication-deficient MVA strain). It is, however, preferred that the expression vectors are ones which are able to express the TCR in a CTL following transfection.

A still further aspect of the invention provides a method of killing target cells in a patient which target cells express a polypeptide comprising an amino acid sequence given in the first aspect of the invention, the method comprising the steps of (1) obtaining CTL from the patient; (2) introducing into said cells a polynucleotide encoding a TCR, or a functionally equivalent molecule, as defined above; and (3) introducing the cells produced in step (2) into the patient. Typically, the target cells are malignant haematopoietic cells which express CD45.

It will be appreciated that, with respect to the methods of killing target cells in a patient, it is particularly preferred that the target cells are leukaemia cells or other malignant haematopoietic cells which express CD45.

The CD45 polypeptide comprises the amino acid sequences FLYDVIAST and ALIAFLAFL and KLFTAKLNV and MIWEQKATV and NLSELHPYL and VNLSELHPYL and LLAFGFAFL and YLYNKETKL and TLILDVPPGV and ILYNNHKFT and ILPYDYNRV and YILIHQALV and KLLAFGFAFL and YQYQYTNWSV.

It is particularly preferred if the patients who are treated by the methods of the invention have the HLA-A0201 haplotype. Thus, in a preferred embodiment the HLA haplotype of the patient is determined prior to treatment. HLA haplotyping may be carried out using any suitable method; such methods are well known in the art.

As discussed in detail above, allorestricted CTLs are used. Thus, it is convenient to determine the HLA haplotype of the donor and to use HLA-mismatched donor cells (at least mismatched for the HLA Class which presents the antigen). Thus, for example, if the patient is typed as HLA-A0201− positive, it is preferred that the donor for the CTL is HLA-A0201− negative so that the CTL are allorestricted

Thus, the invention provides a method of treating a patient with a haematopoietic malignancy, the method comprising (1) determining for a given HLA-binding peptide of human CD45 which type of Class I MHC molecule binds the peptide in the patient, or determining for a given Class I MHC molecule of the patient which peptide (or peptides) of human CD45 binds the Class I MHC molecule in the patient, or both, (2) providing an activated CTL which is allogeneic (allorestricted) with respect to the Class I MHC molecule which binds the peptide in the patient and which CTL is specific for the peptide, (3) undertaking a stem cell transplantation of the patient from a donor who is negative for the type of Class I MHC molecule which, in the patient, binds the peptide, and (4) administering the activated CTL of step (2) to the patient.

Preferably, the peptide of CD45 which binds the MHC Class I molecule is one which may be produced by natural processing of the CD45 antigen.

Typically, the haematopoietic malignancy is leukaemia.

In step (1) determination of which type of Class I MHC molecule binds a particular CD45 HLA-binding peptide may be determined empirically for the patient in question using well known methods. Alternatively, a look-up table for which HLA molecules bind which particular CD45 peptides may readily be generated, for example, Example 1 shows a list of peptides which bind HLA-A0201. In that case, which type of Class I MHC molecule binds the particular peptide can be determined simply by determining whether the patient has the type of Class I MHC molecule to which the particular peptide is known to bind. Determining the type of Class I molecule can be by DNA analysis as is well known in the art. By way of example, the peptide FLYDVIAST binds HLA-A0201 and for this peptide if a patient is shown to have an HLA-A0201 allele the patient is expected to bind this peptide with this MHC molecule.

In step (2), the allogeneic (allorestricted) activated CTL may be produced using the method described above. The activated CTL may conveniently be ones which are known to be peptide specific and for which the HLA genotype has been determined. Thus, the CTL may be selected from a library of CTL which are classified with respect to their peptide specificity and HLA genotype.

The activated CTL administered in step (4) are believed to be able to destroy any residual malignant haematopoietic cells (and also any of the patient's original normal haematopoietic cells) but not the transplanted cells given in step (3).

The invention includes in particular the use of the peptides of the invention (or polynucleotides encoding them) and to activate CTL from healthy donors (MHC mismatched) in vitro followed by adoptive therapy.

The invention will now be described in more detail by reference to the following Figures and Examples in which:

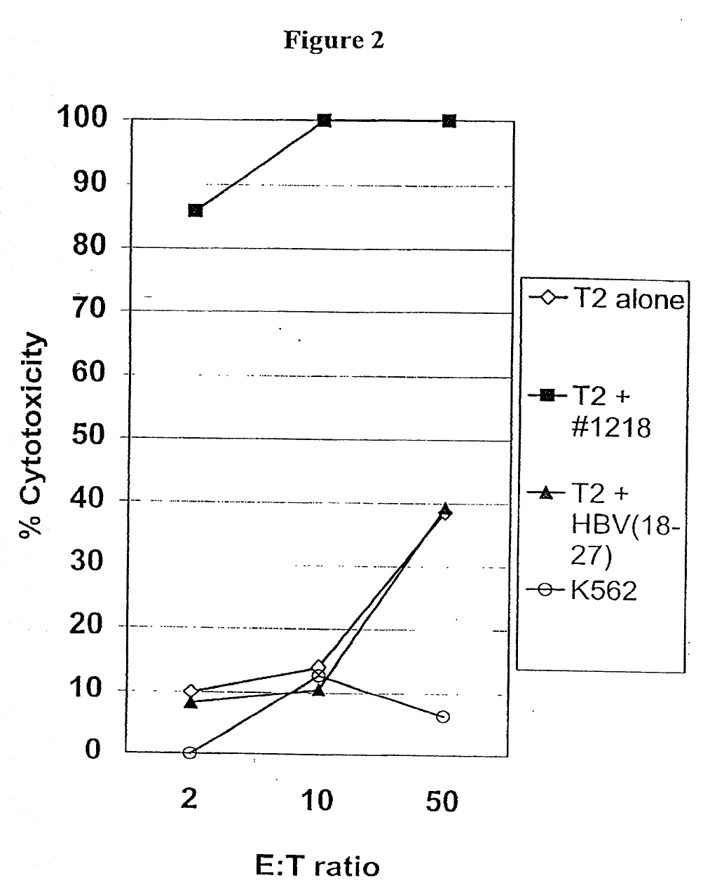

FIG. 1 shows the result of an assay to measure HLA-A0201 binding of peptides. Peptide binding was measured using the T2 assay that is based on the ability of peptides to stabilise HLA-A0201 expression in the TAP-deficient T2 cells. The peptide 1218 (FLYDVIAST) was one of the best binders and was used to stimulate CTL responses (see FIG. 2).

FIG. 2 shows the specificity of 3 CTL lines generated against the CD45-derived peptide 1218. Peptide-specific CTL were isolated from HLA-A0201 negative donors using the method described in WO 97/26328. Three peptide-specific lines were established and found to show specific killing of T2 target cells presenting peptide 1218 but not targets presenting control HLA-A0201− binding peptides (HBV(18-27)). The NK target cells K562 were not killed by these CTL.

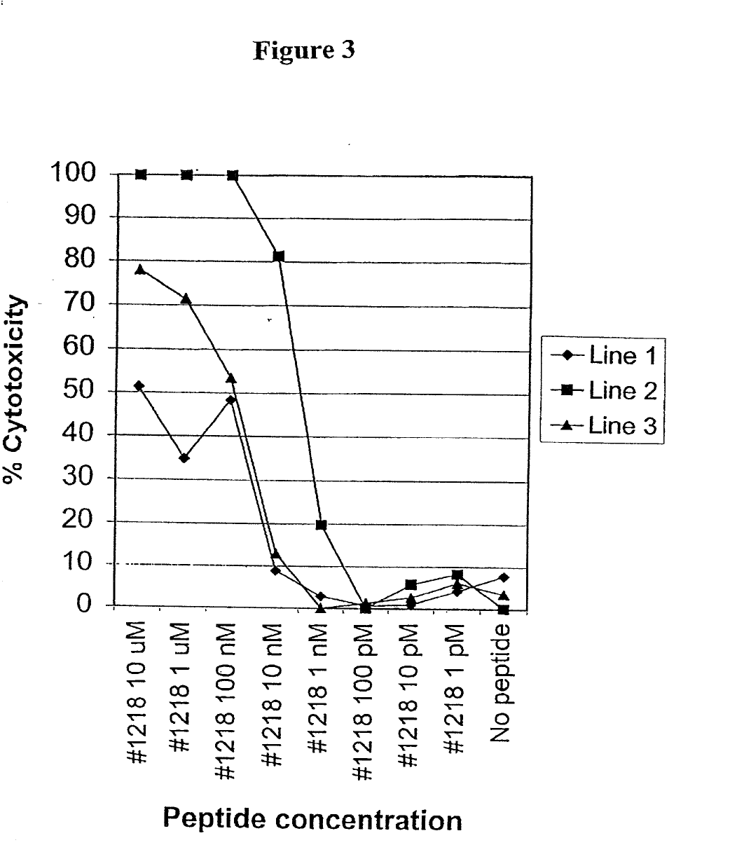

FIG. 3 shows the sensitivity of 3 CTL lines generated against the CD45-derived peptide 1218. The sensitivity of the three CTL lines was tested using T2 target cells coated with decreasing doses of peptide 1218. CTL line 2 was the most sensitive line and used in subsequent experiments.

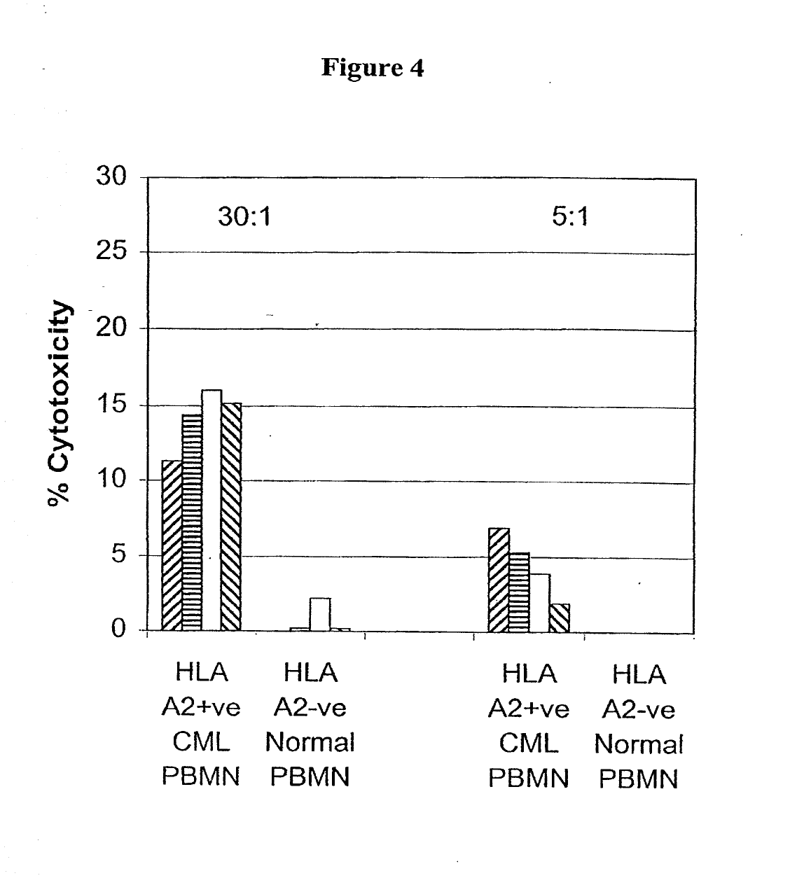

FIG. 4 shows the killing of fresh peripheral blood mononuclear cells (PBMC) from CML (chronic myeloid leukaemia) patients by CTL specific for peptide 1218. CTL line 2 was used to test the killing activity against fresh PBMC from HLA-A0201 positive CML patients. The CTL were also tested against PBMC from HLA-A0201− negative individuals. Effector to target ratio was 30:1 and 5:1.

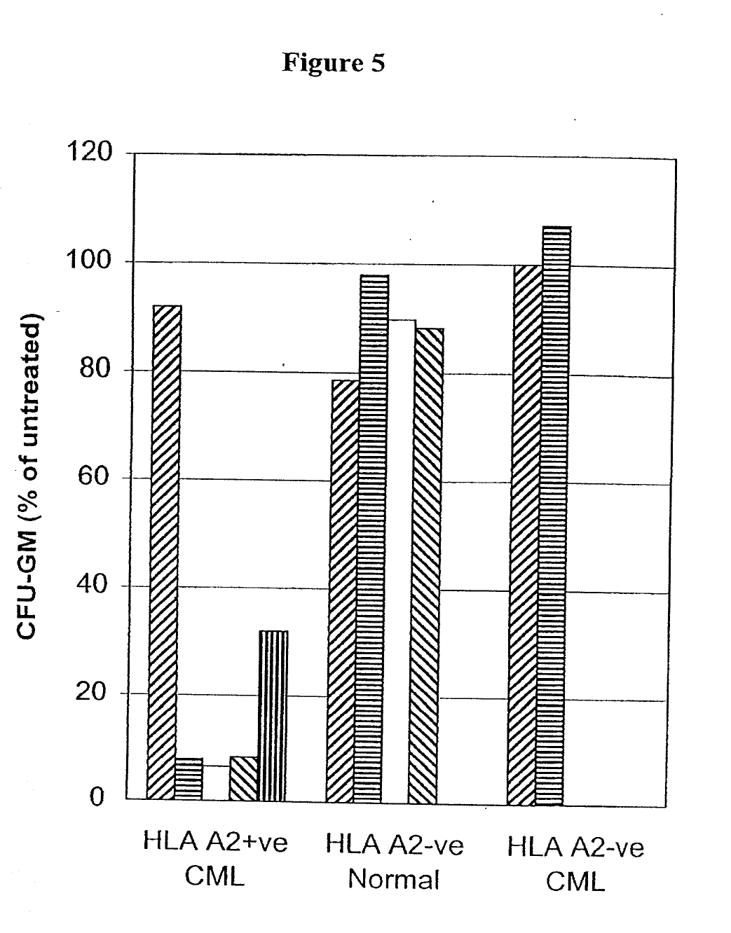

FIG. 5 shows the killing of clonogenic progenitor cells by CTL specific for peptide 1218. CTL line 2 was tested in colony forming assays. The CTL killed most of the colony forming progenitors from HLA-A0201 positive CML patients but not from HLA-A0201 negative normal individuals or CML patients. Purified CD34 progenitor cells were exposed to Cit for 4 hours followed by plating of the treated and control cells to measure the CFU-GM numbers. Shown is the number of colonies in treated CD34 cells relative to the number of colonies in untreated CD34 cells. Treatment eliminated most colonies in CD34 cells of HLA-A0201 positive CML patients.

### EXAMPLE 1

### HLA-A0201− binding Peptides from Human CD45

Sixteen peptides of the CD45 molecule were selected and 14 of them we tested in HLA-A0201 binding assays as described in the legend to FIG. 1. Twelve of the peptides showed binding activity (see FIG. 1).

| | |
|---|---|
| CD45 peptides | |
| huCD45/1218 | FLYDVIAST |
| huCD45/576 | ALIAFLAFL |
| huCD45/244 | KLFTAKLNV |
| huCD45/737 | MIWEQKATV |
| huCD45/919 | NLSELHPYL |
| huCD45/918 | VNLSELHPYL |
| huCD45/7 | LLAFGFAFL |
| huCD45/237 | YLYNKETKL |
| huCD45/293 | LILDVPPGV |
| huCD45/292 | TLILDVPPGV |
| huCD45/369 | ILYNNHKFT |
| huCD45/684 | ILPYDYNRV |
| huCD45/900 | YILIHQALV |
| huCD45/304 | FQLHDCTQV |
| huCD45/6 | KLLAFGFAFL |
| huCD45/1116 | YQYQYTNWSV |

The underlined peptides (huCD45/293 and huCD45/304) were not tested for binding activity.

### EXAMPLE 2

### Stimulation of CTL Responses by Peptide 1218

The Peptide 1218 was one of the best binders and was used to stimulate CTL responses. Peptide-specific CTL were isolated from HLA-A0201 negative donors using the method described in WO 97/26328. Three peptide-specific lines were established and found to show specific killing of T2 target cells presenting peptide 1218 but not targets presenting control peptides. The NK target cells K562 were not killed by these CTL (FIG. 2).

The sensitivity of the three CTL lines was tested using T2 target cells coated with decreasing doses of peptide 1218. CTL line 2 was the most sensitive line (FIG. 3).

CTL line 2 was used to test the killing activity against fresh leukaemic cells from HLA-A0201 positive CML patients. The CTL showed killing of these CML cells but not of normal cells from HLA-A0201− negative donors (FIG. 4).

Finally, CTL line 2 was tested in colony forming assays. The CTL killed most of the colony forming progenitors from HLA-A0201+ CML patients but not from HLA-A0201− individuals (FIG. 5).

These data show that CD45 has at least 12 peptides that can bind to HLA-A0201. Peptide 1218 can induce peptide specific CTL from HLA-A0201 negative donors. The CTL which show 1218 peptide specificity using T2 target cells also kill HLA-A0201 positive CML cells.

We have screened 14 candidate peptides from CD45 for binding to HLA-A201 in T2 binding assays. Using one of the best binding peptides (p1218), we can reliably generate peptide-specific, allorestricted CTL. Tree p1218 specific CTL lines show potent cytotoxicity against HLA A2+ve (C1RA2) but not A2−ve (WS29 or K562) hematopoietic cell lines, although some allreactivity against A2+ve, non-hematopoietic targets was observed. p1218 line 2, which had the highest avidity in peptide tritration assays, showed significant cytotoxicity against PGMN in 4/5HLA A2+ve patients with CML, including 1 in myeloid blast crisis, but 0/4 HLA A2−ve normal controls. Treatment of CD34+ve PBMN/BMMN with p1218 specific CTL inhibited CFU-GM colony formation by >90% in 4/5 HLA A2+ve CML patients without significant inhibition in 5/5 HLA A2−ve normal controls, demonstrating that p1218-specific CTL have potent activity against leukemic progenitors. These data suggest that adoptive immunotherapy with allorestricted CTL directed against CD45 epitopes may be useful in restoring the graft-versus-leukemia effect post haplo-identical transplantation.

### EXAMPLE 3

### CTL Against Peptide 1218 Can Kill Leukaemia Cells but not Fibroblasts

Allo-restricted CTL specific for the CD45-derived peptide, P1218, were tested in 4-hour chromium release assays against the indicated target cells at an effector:target cell ratio (E:T) of 30, 5 and 1. The CTL killed A2− positive leukaemia cells, whilst fibroblasts were killed only after pulsing with the P1218 peptide. A2 negative PBMC were not killed by the CTL.

% Cytotoxicity

| | Patient 1 | Patient 2 | Patient 3 | Patient 4 | Mean +/− SD |
|---|---|---|---|---|---|
| E:T = 30 | | | | | |
| HLA A2 + ve AML blasts | 19.2 | 37.8 | 28.8 | 10 | 24.0 +/− 12.0 |
| HLA A2 + ve CML PBMN | 11.3 | 14.4 | 16 | 15.2 | 14.2 +/− 2.0 |
| HLA A2 − ve Normal PBMN | 0 | 0.2 | 2.2 | 0.2 | 0.7 +/− 1.0 |
| HLA A2 + ve fibroblasts | 0 | 3 | 1.9 | 0 | 1.2 +/− 1.5 |
| HLA A2 + ve fibroblasts + P1218 | 28.2 | 34.1 | 38.1 | 12.6 | 28.3 +/− 11.2 |
| E:T = 5 | | | | | |
| HLA A2 + ve AML blasts | 10 | 21.4 | 24.6 | 8.9 | 16.2 +/− 7.9 |
| HLA A2 + ve CML PBMN | 6.9 | 5.3 | 3.9 | 1.9 | 4.5 +/− 2.1 |
| HLA A2 − ve Normal PBMN | 0 | 0 | 0 | 0 | 0 +/− 0 |
| HLA A2 + ve fibroblasts | 0 | 7.2 | 0 | 0 | 1.8 +/− 3.6 |
| HLA A2 + ve fibroblasts + P1218 | 20.1 | 30.8 | 31.5 | 9.2 | 22.9 +/− 10.5 |
| E:T = 1 | | | | | |
| HLA A2 + ve AML blasts | 0 | 17 | 13.6 | 0.9 | 7.9 +/− 8.7 |
| HLA A2 + ve CML PBMN | 0 | 0 | 0 | 0 | 0 +/− 0 |
| HLA A2 − ve Normal PBMN | 0 | 0 | 0 | 0 | 0 +/− 0 |
| HLA A2 + ve fibroblasts | 0 | 3.1 | 1.2 | 0 | 1.1 +/− 1.5 |
| HLA A2 + ve fibroblasts + P1218 | 14.4 | 23.2 | 31.6 | 9.7 | 19.7 +/− 9.7 |

### EXAMPLE 4

### Production of Activated Cytotoxic Lymphocytes (CTL) Using Class I Molecules and the CD45 Peptide Antigen FLYDVIAST and Their Administration

Activated cytotoxic T lymphocytes (CTLs) are produced using HLA-A2 Class I molecules and the nonamer peptide from CD45: FLYDVIAST.

The method described in PCT patent application WO 93/17095 is used to make the CTLs. Cells with an endogenous defect in the peptide loading of HLA Class I molecules are used as stimulator cells. For example, human T2 cells, murine RMA-S/A2 cells or Drosophila cells transfected with human HLA-A0201, B7.1 and ICAM-1. The HLA-A0201 molecules expressed in these cells can be conveniently loaded with exogenously supplied peptides.

The peptide is synthesised on an Applied Biosystems synthesiser, A-BI 431A (Foster City, Calif., USA) and subsequently purified by HPLC.

As is described in detail in WO 93/17095, in order to optimize the in vitro conditions for the generation of specific cytotoxic T cells, the culture of stimulator cells is maintained in an appropriate medium. The stimulator cells are T2 or RMA-S/A2 or Drosophila cells as described in WO 93/17095.

Prior to incubation of the stimulator cells with the cells to be activated, eg precursor CD8 cells, an amount of antigenic peptide is added to the stimulator cell culture, of sufficient quantity to become loaded onto the human Class I molecules to be expressed on the surface of the stimulator cells. A sufficient amount of peptide is an amount that will allow about 200, and preferably 200 or more, human Class I MHC molecules loaded with peptide to be expressed on the surface of each stimulator cell. The stimulator cells are typically incubated with >1 μg/ml peptide.

Resting or precursor CD8 cells are then incubated in culture with the appropriate stimulator cells for a time period sufficient to activate the CD8 cells. The CD8 cells shall thus be activated in an antigen-specific manner. The ratio of resting or precursor CD8 (effector) cells to stimulator cells may vary from individual to individual and may further depend upon variables such as the amenability of an individual's lymphocytes to culturing conditions. The lymphocyte:stimulator cell (Drosophila cell) ratio is typically in the range of about 2:1 to 100:1. For example, 3×10⁷ human PBL and 3×10⁶ live Drosophila cells are admixed and maintained in 20 ml of RPMI 1640 culture medium.

The effector/stimulator culture are maintained for as long a time as is necessary to stimulate a therapeutically usable or effective number of CD8 cells. This usually requires several rounds of bulk stimulation followed by limiting dilution cultures to isolate peptide-specific CTL lines.

Effective, cytotoxic amounts of the activated CD8 cells can vary between in vitro and in vivo uses, as well as with the amount and type of cells that are the ultimate target of these killer cells between about 1×10⁶ and 1×10¹² activated CTL are used for adult humans.

Methods of re-introducing cellular components are used such as those exemplified in U.S. Pat. No. 4,844,893 to Honsik et al and U.S. Pat. No. 4,690,915 to Rosenberg. For example, administration of activated CD8 cells via intravenous infusion is appropriate.

1. A peptide comprising an HLA-binding peptide of human CD45 polypeptide or a portion or variant of said peptide provided that the peptide is not the intact human CD45 polypeptide.

2. A peptide according to claim 1 wherein the peptide comprises at least one amino acid sequence selected from the group consisting of FLYDVIAST, ALIAFLAFL, KLFTAKLNV, MIWEQKATV, NLSELHPYL, VNLSELHPYL, LLAFGFAFL YLYNKETKL, LILDVPPGV, TLILDVPPGV, ILYNNHKFT, ILPYDYNRV, YILIHQALV, FQLHDCTQV, KLLAFGFAFL, YQYQYTNWSV, and portions and variants of any of these.

3. A peptide according to claim 1 wherein the peptide or the said portion or variant thereof is capable of binding to HLA-A0201.

4. A peptide according to claim 3 wherein when bound to HLA-A0201 the peptide-bound HLA-A0201 is capable of eliciting the production of a cytotoxic T lymphocyte (CTL) which recognizes a cell which expresses a polypeptide comprising the given amino acid sequence.

5. A peptide according to claim 1 wherein the peptide includes non-peptide bonds.

6. A peptide comprising at least one amino acid sequence selected from the group consisting of FLYDVIAST, ALIAFLAFL, KLFTAKLNV, MIWEQKATV, NLSELHPYL, VNLSELHPYL, LLAFGFAFL, YLYNKETKL, LILDVPPGV, TLILDVPPGV, ILYNNHKFT, ILPYDYNRV, YILIHQALV, FQLHDCTQV, KLLAFGFAFL, and YQYQYTNSWV.

7. A peptide according to claim 1 forming a polypeptide fusion molecule which comprises an HLA heavy chain molecule joined via a flexible linker to an HLA-binding peptide of CD45 such that the HLA-binding peptide is able to occupy the peptide-binding groove of the HLA molecule.

8. A polynucleotide encoding a peptide according to claim 1 or fusion molecule thereof which comprises an HLA heavy chain molecule joined via a flexible linker to an HLA-binding peptide of CD45 such that the HLA-binding peptide is able to occupy the peptide-binding groove of the HLA molecule.

9. A polynucleotide according to claim 8 which is DNA.

10. The polynucleotide according to claim 8 further comprising an expression vector capable of expressing the peptide.

11. A host cell comprising a polynucleotide according to claim 8 alone or in an expression vector.

12. A method of producing a peptide according to claim 1 or fusion molecule thereof which comprises an HLA heavy chain molecule joined via a flexible linker to an HLA-binding peptide of CD45 such that the HLA-binding peptide is able to occupy the peptide-binding groove of the HLA molecule comprising culturing the host cell according to claim 11 and obtaining the peptide from the host cell or its culture medium.

13. A kit of parts comprising a peptide according to claim 1 and antigen presenting cell.

14. A kit of parts according to claim 13 wherein the antigen presenting cell is a cell defective in, or lacks, the expression of the TAP peptide transporter.

15. A kit of parts according to claim 14 wherein the cell is any one of the T2 cell, an RMA-S cell or a Drosophila cell.

16. An antigen-presenting cell wherein its MHC Class I molecules are loaded with a peptide according to claim 1.

17. A cell according to claim 16 which is defective in, or lacks, the expression of the TAP peptide transporter.

18. A cell according to claim 17 selected from the group consisting of a T2 cell, an RMA-S cell and a Drosophila cell.

19. A method for producing activated cytotoxic T lymphocytes (CTL) in vitro, the method comprising contacting in vitro CTL, which antigen-loaded human class I MHC molecules expressed on the surface of a suitable antigen-presenting cell for a period of time sufficient to activate, in an antigen specific manner, said CTL, wherein the antigen is a peptide according to claim 1.

20. A method according to claim 19 wherein the CTL and the antigen-presenting cell are allogeneic with respect to the class I MHC molecule that is presenting peptides of CD45.

21. A method according to claim 19 wherein the antigen is loaded onto class I MHC molecules expressed on the surface of a suitable antigen-presenting cell by contacting a sufficient amount of the antigen with an antigen presenting cell wherein before contact the class I MHC molecules of the antigen-presenting cell are substantially unoccupied and after contact the class I MHC molecules are substantially fully occupied.

22. A method according to claim 19 wherein the antigen-presenting cell comprises an expression vector which expresses a peptide according to claim 10.

23. A method to any one of claim 19 wherein the class I MHC molecule in HLA-A0201.

24. Activated cytotoxic T lymphocytes (CTL) obtainable by the method according to claim 19.

25. Activated cytotoxic T lymphocytes (CTL) which selectively recognise a cell which expresses a polypeptide comprising an amino acid sequence given in claim 1.

26. Activated cytotoxic T lymphocytes (CTL) which selectively recognise a malignant cell which expresses CD45.

27. A T-cell receptor (TCR) which recognises a cell which expresses a polypeptide comprising an amino acid sequence given in claim 1.

28. A T-cell receptor (TCR) or a functionally equivalent molecule to the TCR which recognises a malignant haematopoietic which expresses CD45.

29. A polynucleotide encoding a T cell receptor (TCR) selected from the group consisting of a T-cell receptor (TCR) which recognises a cell which expresses a polypeptide comprising an amino acid sequence given in claim 1 and a T-cell receptor (TCR) or a functionally equivalent molecule to the TCR which recognises a malignant haematopoietic which expresses CD45

30. An expression vector capable of expressing a T cell receptor (TCR) selected from the group consisting of a T-cell receptor (TCR) which recognises a cell which expresses a polypeptide comprising an amino acid sequence given in claim 1 and a T-cell receptor (TCR) or a functionally equivalent molecule to the TCR which recognises a malignant haematopoietic which expresses CD45.

31. A method of killing target cells in a patient which target cells express a polypeptide comprising an amino acid sequence given in claim 1, the method comprising administering to the patient an effective number of cytotoxic T lymphocytes (CTL) selected from the group consisting of activated cytotoxic T lymphocytes (CTL) obtainable by the method according to claim 19, activated cytotoxic T lymphocytes (CTL) which selectively recognise a cell which expresses a polypeptide comprising an amino acid sequence given in claim 1, and activated cytotoxic T lymphocytes (CTL) which selectively recognise a malignant cell which expresses CD45.

32. A method according to claim 31 wherein the patient has undergone an allogeneic stem cell transplantation.

33. A method according to claim 31 wherein the target cells are cancer cells.

34. A method according to claim 33 wherein the cancer is a leukaemia which expresses the CD45 polypeptide.

35. A method of treating a patient with a haematopoietic malignancy, the method comprising (1) determining for a given HLA-binding peptide of human CD45 which type of Class I MHC molecule binds the peptide in a patient, or determining for a given Class I MHC molecule of the patient which peptide (or peptides) of human CD45 binds the Class I MHC molecule in the patient, or both, (2) providing an activated CTL which is allogeneic (allorestricted) with respect to the Class I MHC molecule which binds the peptide in the patient and which CTL is specific for the peptide, (3) undertaking a stem cell transplantation of the patient from a donor who is negative for the type of Class I MHC molecule which, in the patient, binds the peptide, and (4) administering the activated CTL of step (2) to the patient.

36. A method according to claim 35 wherein the type of Class I MHC molecule is determined by DNA analysis.

37. A method according to claim 35 wherein in step (2) the activated CTL are produced by the method of claim 19 wherein the CTL are allogenic (allorestricted) with respect to the Class I MHC molecule which presents the peptide in the patient.

38. A method according to claim 35 wherein in step (2) the activated CTL are selected from a library of CTL.

39. A library of activated CTL wherein each member of the library (1) recognises a CD45 peptide when presented by a particular, recorded HLA and (2) has its HLA haplotype recorded.

40. A library of HLA-binding peptides of human CD45 polypeptide wherein for each member of the library the type of HLA molecule it binds is recorded.

41. A library of antigen presenting cells each loaded with an HLA-binding peptide of human CD45 polypeptide wherein for each member of the library the identity of the peptide is recorded and, optionally, the HLA haplotype of the antigen presenting cell.
